(19) United States
(12) Patent Application Publication
(10) Pub. No.: US 2025/0051803 A1
(43) Pub. Date: Feb. 13, 2025

(54) DNA POLYMERASE-MEDIATED GENOME EDITING

(71) Applicant: Wuhan University, Wuhan, Hubei (CN)

(72) Inventors: Hao YIN, Wuhan, Hubei (CN); Ying ZHANG, Wuhan, Hubei (CN); Shuhan LU, Wuhan, Hubei (CN); Jinlin WANG, Wuhan, Hubei (CN)

(21) Appl. No.: 18/719,240

(22) PCT Filed: Dec. 14, 2022
(86) PCT No.: PCT/CN2022/138921

§ 371 Date: Jun. 12, 2024

(30) Foreign Application Priority Data
Dec. 15, 2021 (WO) PCT/CN2021/138430

(51) Int. Cl. C12N 15/90; C12N 9/12; C12N 9/22; C12N 15/11

(52) U.S. Cl. CPC C12N 15/907; C12N 9/1252; C12N 9/22; C12N 15/11; C12N 2310/20

(57) ABSTRACT

Provided are compositions and methods useful for inserting or replacing a nucleic acid fragment to a target genome sequence. Unlike the conventional prime editors, the disclosed methods do not require a retro-transcriptase or a pegRNA. Instead, the Cas protein is fused to, or otherwise coupled to, or is present within the same cell as, a DNA polymerase, which uses a single stranded donor DNA to generate the desired insertion sequence.

### BACKGROUND

Genome editing tools can be used to manipulate the genome of cells and living organism and thus have broad interest in life science research, biotechnology, agricultural technology and most importantly disease treatment. A novel CRISPR-based gene editor, called Prime editing (PE) was developed through linking a reverse transcriptase (RT) to a Cas9 nickase. The RT template (RTT) is at the 3′ of the prime editing guide RNA (pegRNA), leading to precise modification of the nicked site. Prime editing is able to mediate all types of base editing, small insertion and deletion without donor DNA, holding great potential for basic research and correction of genetic mutants associated with human diseases.

### SUMMARY

Provided in various embodiments are compositions and methods useful for inserting or replacing a nucleic acid fragment at a target genome sequence. Unlike the conventional prime editing systems, the disclosed methods do not require a retro-transcriptase or a pegRNA. Instead, the Cas protein is fused to, or otherwise coupled to (or even just co-present, e.g., in a cell, with) a DNA polymerase, which uses a single stranded donor DNA (ssDNA) to generate the desired insertion sequence. The newly formed sequence templated by the ssDNA is a double stranded DNA fragment, which can be readily ligated to the other end of the genome sequence left open by the Cas protein.

The conventional prime editing system generates a single stranded DNA from the RNA template, which can only be incorporated into the genome by virtue of its homology to the original genome sequence. Such a conventional technology, therefore, can only afford to insert very short sequences or engender mutations. By contrast, the presently disclosed technology does not require the ssDNA to be homologous to the genomic sequence (except for a short 3′ portion to hybridize to a released genomic sequence flap to initiate DNA replication). Accordingly, the instant technology can insert any sequence of choice, and of large length, such as hundreds of base pairs.

One embodiment of the present disclosure provide a molecule comprising (a) a Cas protein and (b) a DNA polymerase, wherein the Cas protein is fused to the DNA polymerase or is coupled to the DNA polymerase through a covalent or ionic interaction, directly or indirectly.

In some embodiments, the Cas protein is selected from the group consisting of Cas9, Cas12, Cas13 and Cas14. In some embodiments, the Cas protein is Cas9. In some embodiments, the Cas9 is selected from the group consisting of SpyCas9, SaCas9, NmeCas9, FnCas9 and CjCas9. In some embodiments, the Cas9 is a nickase, preferably Cas9 H840A.

In some embodiments, the DNA polymerase is selected from the group consisting of eukaryotic DNA polymerase family A, B, C, X and Y such as DNA polymerase α, γ, β, λ, ε, δ, κ, η, ξ, ι, θ, μ, σ, ν, Rev1, TdT, telomerase and human codon-optimized prokaryotic DNA polymerase from family Pol I, Pol II, Pol III, Pol IV, Pol V and Family D, including E. coli DNA polymerase I, DNA polymerase III, engineered DNA polymerase from virus and phage, such as codon-optimized Bacteriophage T4 DNA polymerase.

In some embodiments, the molecule further comprises an accessory protein replication factor C (RF-C), a proliferating cell nuclear antigen (PCNA) or a DNA helicase.

In some embodiments, the molecule further comprises a single guide RNA (sgRNA). In some embodiments, the molecule further comprises a single stranded DNA (ssDNA).

In some embodiments, the Cas protein is fused to the DNA polymerase. In some embodiments, the Cas protein is located at the N-terminal side of the DNA polymerase, or at the C-terminal side of the DNA polymerase.

Also provided, in one embodiments, is a method for introducing a foreign nucleotide sequence into a target nucleotide, comprising contacting, in a cell, the target nucleotide with a Cas protein fused or coupled to (or co-present with in the cell) a DNA polymerase, a single guide RNA (sgRNA) comprising a spacer complementary to a protospacer in the target nucleotide, a donor single stranded DNA (ssDNA) that is complementary to a portion of the target nucleotide on the opposite strand of the protospacer, and further encodes the foreign nucleotide sequence.

Also provided, in one embodiments, is a method for introducing a foreign nucleotide sequence into a target nucleotide, comprising contacting, in a cell, the target nucleotide with a Cas protein fused or coupled to a DNA polymerase, (a) a first single guide RNA (sgRNA) comprising a first spacer complementary to a first protospacer in the target nucleotide, a first donor single stranded DNA (ssDNA) that is complementary to a first portion of the target nucleotide on the opposite strand of the first protospacer, and further encodes a first portion of the foreign nucleotide sequence; and (b) a second single guide RNA (sgRNA) comprising a second spacer complementary to a second protospacer in the target nucleotide, a second donor single stranded DNA (ssDNA) that is complementary to a second portion of the target nucleotide on the opposite strand of the second protospacer, and further encodes the remaining portion of the foreign nucleotide sequence.

In some embodiments, each of the first ssDNA and the second ssDNA further comprise a 5′ fragment complementary to each other.

In some embodiments, each 5′ fragment is of length of 1 to 50 bases, 2 to 40 bases 3 to 30 bases, 4 to 25 bases, 5 to 20 bases, 5 to 15 bases, or 5 to 10 bases.

In some embodiments, each ssDNA further comprises a spacer or a protospacer adjacent motif (PAM) 5′ to the portion that encodes the foreign nucleotide sequence.

In some embodiments, the cell further includes a third sgRNA that recognizes the spacer or the PAM on each of the ssDNA.

In some embodiments, each complementary portion of the target nucleotide and the corresponding protospacer are on opposite strands of the target nucleotide and are within 10000 base pairs from each other, or preferably within 5000, 1000, 500, 100, 90, 80, 70, 60, 50, 40, 30, 20, 15 or 10 base pairs from each other.

In some embodiments, the complementary portion is of length of 1 to 50 bases, 2 to 40 bases 3 to 30 bases, 4 to 25 bases, 5 to 20 bases, 5 to 15 bases, or 5 to 10 bases.

In some embodiments, the Cas protein is selected from the group consisting of Cas9, Cas12, Cas13 and Cas14. In some embodiments, the Cas protein is Cas9. In some embodiments, the Cas9 is selected from the group consisting of SpyCas9, SaCas9, NmeCas9, FnCas9 and CjCas9. In some embodiments, the Cas9 is a nickase, preferably Cas9 H840A.

In some embodiments, the DNA polymerase is selected from the group consisting of eukaryotic DNA polymerase family A, B, C, X and Y such as DNA polymerase α, γ, β, λ, ε, δ, κ, η, ξ, ι, θ, μ, σ, ν, Rev1, TdT, telomerase and human codon-optimized prokaryotic DNA polymerase from family Pol I, Pol II, Pol III, Pol IV, Pol V and Family D, including E. coli DNA polymerase I, DNA polymerase III, engineered DNA polymerase from virus and phage, such as codon-optimized Bacteriophage T4 DNA polymerase.

In some embodiments, the Cas protein or DNA polymerase is further fused to or coupled to an accessory protein replication factor C (RF-C), a proliferating cell nuclear antigen (PCNA) or a DNA helicase.

In some embodiments, each ssDNA is provided as or released from a linear single stranded DNA, a linear double stranded DNA, a DNA/RNA hybrid, a single stranded DNA vector, a circular single stranded DNA, a circular double-stranded DNA, or a circular DNA/RNA hybrid.

In some embodiments, each ssDNA is modified with a group selected from the group consisting of phosphoryl, biotin, digoxigenin, amino, thiol, phosphorthioate, methyl, and 2′-O-methyl-3′-phosphonoacetate (MP).

In some embodiments, each ssDNA is provided alone, or covalently or non-covalently coupled to the Cas protein, the DNA polymerase, or the sgRNA.

In some embodiments, each ssDNA is bound to a DNA-binding protein preferably fused or coupled to the Cas protein or the DNA polymerase.

In some embodiments, the Cas protein is fused to the DNA polymerase.

In some embodiments, the Cas protein is located at the N-terminal side of the DNA polymerase, or at the C-terminal side of the DNA polymerase.

In some embodiments, the cell is a eukaryotic cell, preferably a mammalian cell, such as a human cell.

### BRIEF DESCRIPTION OF THE DRAWINGS

FIG. 1-6 illustrate various embodiments of the present technology.

FIG. 7 shows the sequencing results confirming insertion of a sequence.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein”, “amino acid chain” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

### DNA Polymerase-Mediated Genome Editing

The present disclosure provides compositions and methods for improved genome editing. The instantly disclosed technology, also referred to as a “DNA polymerase-mediated genome editing,” has similar or even improved editing efficiency as compared to the conventional prime editing, but does not require a retro-transcriptase (RT) or a large prime editing guide RNA (pegRNA).

One embodiment of the technology is illustrated in FIG. 1. A fusion protein is provided that includes a Cas protein and a DNA polymerase. Alternatively, the Cas protein and the DNA polymerase are prepared separately and then coupled together with known technologies, such as protein-protein interaction, disulfide bonds, conjugation, or through recruitment by RNA-protein interaction, or by chance to present in the same position of the genome.

In some embodiments, the Cas protein is a Cas9, such as SpyCas9, SaCas9, NmeCas9, FnCas9 and CjCas9, without limitation. In some embodiments, the Cas protein is a Cas9 nickase. An example nickase is Cas9 H840A. The Cas9 enzyme contains two nuclease domains that can cleave DNA sequences, a RuvC domain that cleaves the non-target strand and a HNH domain that cleaves the target strand. The introduction of a H840A substitution in Cas9, through which the histidine residue at 840 is replaced by an alanine, inactivates the HNH domain. With only the RuvC functioning domain, the catalytically impaired Cas9 introduces a single strand nick, hence a nickase.

In some embodiments, the Cas protein is Cas9, Cas12, Cas13 or Cas14.

Non-limiting examples of DNA polymerase include members of the eukaryotic DNA polymerase family A, B, C, X and Y such as DNA polymerase α, γ, β, λ, ε, δ, κ, η, ξ, ι, θ, μ, σ, ν, Rev1, TdT, telomerase and human codon-optimized prokaryotic DNA polymerase from family Pol I, Pol II, Pol III, Pol IV, Pol V and Family D, including E. coli DNA polymerase I, DNA polymerase III. Engineered DNA polymerase from virus and phage can also be selected, such as codon-optimized Bacteriophage T4 DNA polymerase.

In some embodiments, the accessory protein replication factor C (RF-C), the proliferating cell nuclear antigen (PCNA) or DNA helicase can be fused or otherwise coupled to the Cas protein or the DNA polymerase to improve the activity of DNA polymerase.

Methods of using the fusion (or coupled/conjugated) molecules for genome editing are also illustrated in FIG. 1. In addition to the fusion (or coupled/conjugated, recruited or by chance to interact in cells) molecule that includes the Cas protein and the DNA polymerase, a single guide RNA (sgRNA) and a single stranded donor DNA (ssDNA) are also provided.

In the conventional prime editing system, a pegRNA is used that includes, in addition to a single guide RNA (sgRNA), a reverse transcriptase (RT) template sequence and a primer binding site (PBS). The PBS is complementary to the guide sequence (or “spacer”) in the sgRNA, but is typically a few nucleotides shorter. When the guide sequence binds to the target genome sequence and dissociates the DNA double helix, the PBS binds to the opposite strand and initiates reverse transcription, using the RT template sequence as a template. The RT template can include mutations or small insertions relative to the target genome sequence, but needs to be largely homologous to the target genome sequence.

It is worth noting that the sgRNA used here can optionally include the RT template and/or the PBS as well, but preferably does not include either or both of them. Instead, a donor ssDNA is used as the template (not RT template, but a DNA polymerase template).

Accordingly, in some embodiments, the present composition or method does not include a pegRNA that includes an RT template or primer binding site (PBS).

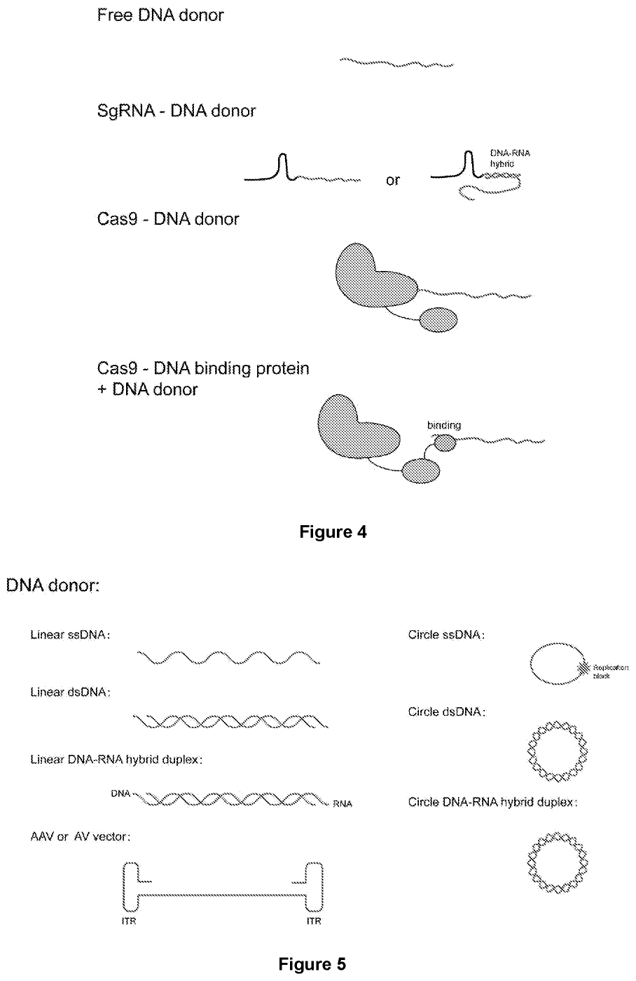

In some embodiments, the sgRNA and the ssDNA are provided separately, and they can both be recruited by the Cas system at the target site. In some embodiments, the sgRNA and the ssDNA are provided as a bound complex or fused nucleic acid. For instance, in FIG. 4, the sgRNA can be chemically conjugated to the ssDNA, or hybridize to the ssDNA (DNA-RNA hybrid). As such, they can be recruited together to the target genome site, potentially increasing editing efficiency.

In another embodiment, the donor ssDNA is coupled to the Cas protein/DNA polymerase fusion protein or complex. In one example illustrated in FIG. 4, the ssDNA is conjugated to the Cas protein directly. In another example, the ssDNA is bound to a DNA-binding protein, which in turn is fused to or coupled to the Cas protein/DNA polymerase fusion protein or complex.

The donor ssDNA can be provided in different manners. A few examples are illustrated in FIG. 5. In one embodiment, the ssDNA is provided as a linear DNA molecule. In another embodiment, the ssDNA is released from a double-stranded DNA (dsDNA) molecule that is originally provided. In another embodiment, the ssDNA is released from a DNA/RNA hybrid molecule. In another embodiment, the ssDNA is provided in a viral vector which, optionally, includes internal repeat sequences for improved stability and durability.

In some embodiments, the ssDNA is generated from a circular ssDNA. In some embodiments, the ssDNA is generated from a circular dsDNA. Still in another embodiment, the ssDNA is provided in the form of a circular DNA/RNA hybrid duplex.

In some embodiments, the donor ssDNA is modified to enhance the binding affinity of DNA and DNA and/or improve donor DNA stability. Example modifications may be with a group selected from the group consisting of phosphoryl, biotin, digoxigenin, amino, thiol, phosphorthioate, methyl, and 2′-O-methyl-3′-phosphonoacetate (MP), and other existing modifications.

The donor DNA may be delivered alone or conjugated with Cas9 or sgRNA in covalent or non-covalent forms. DNA donor can also be delivered fused with a specific DNA sequence and interacts with DNA binding protein fused with Cas9, for example the transcription factors IRF3 bind specific DNA sequences in promotor of IFNβ, TALEN, Zinc-Finger.

In some embodiments, the donor DNA includes a replication block that includes a loop structure or termination sequence to stop the DNA synthesis.

Back to FIG. 1, at step a, the sgRNA helps recruit the Cas protein/DNA polymerase fusion protein/complex to the target genome site given the sgRNA's sequence complementarity with the target site. At step b, the Cas protein cuts open one of the strands in the target DNA, which releases a single stranded DNA “tail” (or flap) that is complementary to a portion of the donor ssDNA. The ssDNA then hybridizes to the released tail (step c) and serves as a template for the DNA polymerase for extending the tail (step d).

Meanwhile, endogenous DNase digests portions of the non-hybridized DNA at the target (step e), facilitating ligation between the newly formed double-stranded insertion (extension based on the donor ssDNA) and the end of the other side of the target DNA (step f). Accordingly, a double stranded sequence corresponding to the template sequence on the donor ssDNA is inserted into the target DNA.

The ssDNA, in some embodiments, includes a portion that is complementary to the “tail” (or flap) that enables the ssDNA to bind to it and initiate DNA replication. The tail is released by the sgRNA/Cas, and is therefore at the opposite strand that the sgRNA binds to. The tail is typically near the protospacer or the protospacer adjacent motif (PAM), such as within 100 bp, 90 bp, 80 bp, 70 bp, 60 bp, 50 bp, 40 bp, 30 bp, 25 bp, 20 bp, 15 bp, 10 bp or 5 bp.

In some embodiments, the hybridization between the tail and the ssDNA is of a length that allows DNA replication to commence. For instance, the complementary portion (between the ssDNA and the target genome) is of a length of 1 to 50 bases, 2 to 40 bases 3 to 30 bases, 4 to 25 bases, 5 to 20 bases, 5 to 15 bases, or 5 to 10 bases.

In a conventional prime editing system, part of the pegRNA serves as a template for the retro-transcriptase, and the extended portion is a RNA/DNA hybrid. Once the RNA is degraded, for the extended single-stranded DNA to incorporate into the genome, it must be homologous to the genome sequence. Therefore, the conventional prime editing systems can only introduce small changes to a target genome, which small changes must be embedded in the homologous sequence.

By contrast, the donor DNA here does not need to be homologous to the target genome sequence (except for a relatively short 3′ portion that hybridizes to the genome to initiate DNA replication, see FIG. 1, step c). This is at least because the newly formed double-stranded DNA fragment can directly join to the other end of the genomic sequence. Therefore, the present technology can insert any sequence or large sizes, which does not need to be homologous to the target genomic sequence.

In some embodiments, the inserted sequence (or the coding region of the ssDNA) is of a size that is at least 1 bp, 5 bp, 10 bp, 20 bp, 30 bp, 40 bp, 50 bp, 80 bp, 100 bp, 150 bp, 200 bp, 250 bp, 300 bp, 400 bp, 500 bp, 600 bp, 700 bp, 800 bp, 900 bp, 1 kb or 10 kb (or in unit of bases for the ssDNA).

### Large Insertion

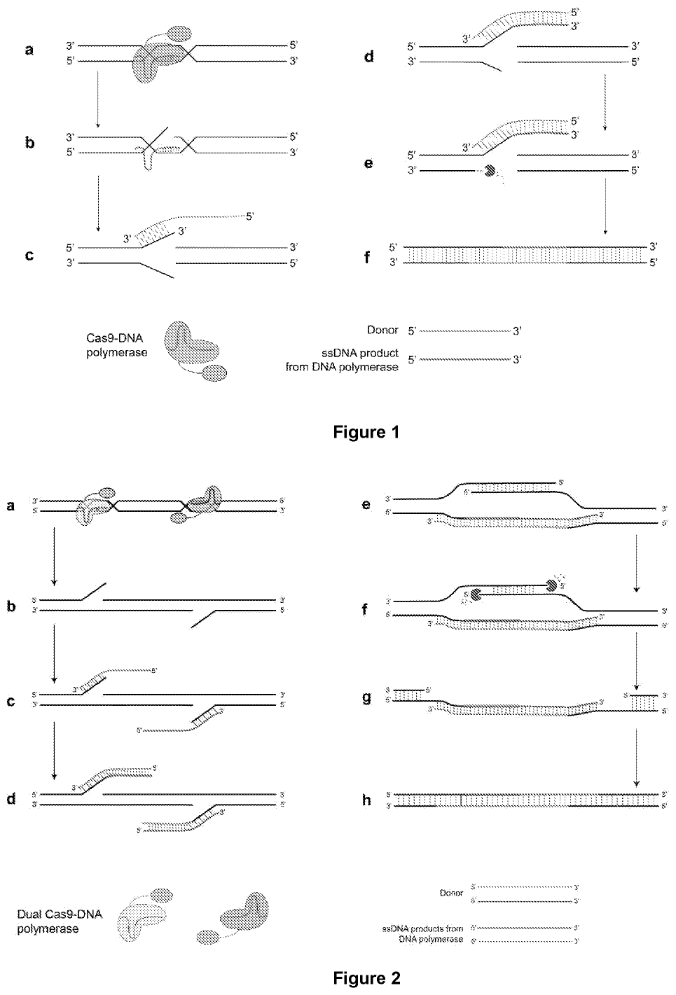

Another embodiment of the present technology allows insertion of a relatively large DNA sequence into a target site. This is illustrated in FIG. 2.

The method entails the use of a pair of Cas protein/DNA polymerase fusions/complexes. Each of them is also provided with a sgRNA, designed to target two proximate sites on a genome sequence. Each sgRNA is then provided with a corresponding donor ssDNA. The corresponding ssDNA includes a region complementary to a tail released from the genome once the sgRNA/Cas protein are recruited to the site and nick it.

Accordingly, as illustrated in FIG. 2, when both Cas protein/DNA polymerase are recruited the proximate sites and subsequently both ssDNA guide the extension of the newly released tails, the newly formed (extended) double stranded fragments can join, resulting in the insertion of small or large fragment encoded, collectively, by both donor ssDNA.

This methodology, therefore, allows the insertion of sequence that is twice as large as what a single Cas protein/DNA polymerase fusion/complex can do.

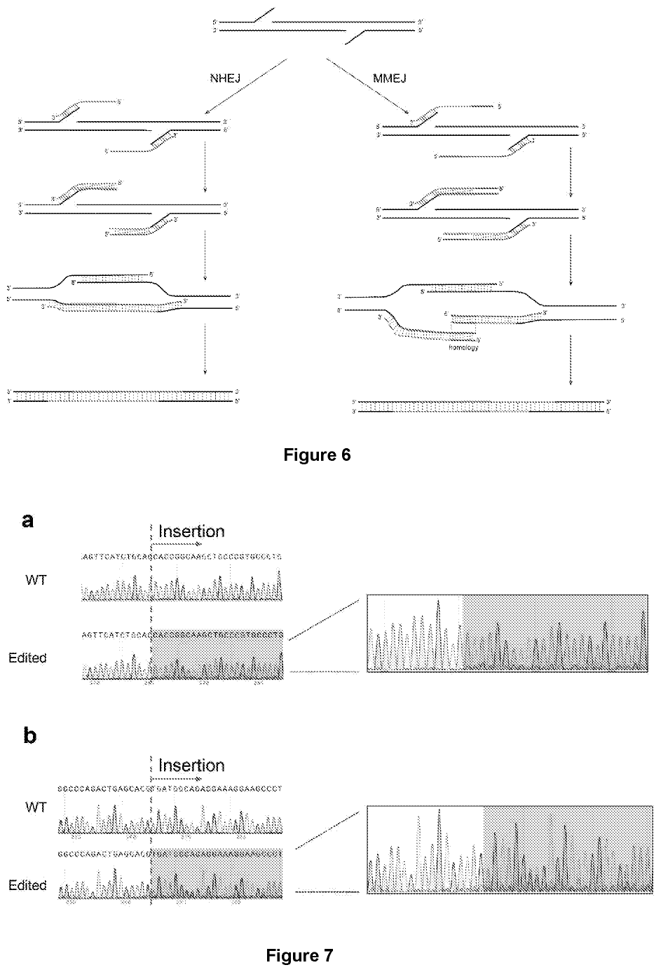

In another embodiment, each of the two donor ssDNA not only includes portions serving as template for extending the genomic sequences, but also includes a distal end that is complementary to each other. Therefore, as illustrated in FIG. 6 (right panel), the newly extended double-stranded fragments have complementary sequences at their ends, allowing microhomology-mediated end joining (MMEJ), which is contemplated to have improved efficiency and precision. The left panel of FIG. 6 illustrates joining by non-homologous end joining (NHEJ) with blunt ends.

In some embodiments, this additional fragment (at the 5′ end of each ssDNA) that is complementary to each other has a length of 1 to 50 bases, 2 to 40 bases 3 to 30 bases, 4 to 25 bases, 5 to 20 bases, 5 to 15 bases, or 5 to 10 bases, without limitation.

### Joining of Sticky Ends

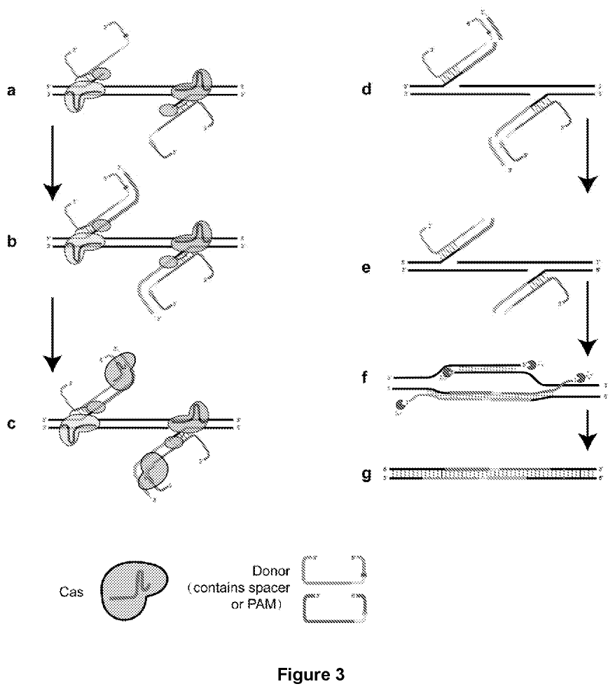

In another embodiment of the DNA polymerase-mediated genome editing, sticky ends from both ssDNA-guided extensions are formed, facilitating ligation of the ends. This embodiment is illustrated in FIG. 3.

Similar to the process of FIG. 2, the embodiment of FIG. 3 employs ssDNA that further includes, in addition to the complementary portion to the genome sequence and the extension template (insertion sequence), (a) an extra fragment at the 5′, and (b) a spacer or PAM sequence between (a) and extension template.

Also further used is a third sgRNA capable of recognizing the (b) spacer and/or PAM sequence(s) on the ssDNA. Therefore, at step c, after both ssDNA successfully extended the genomic sequences, the extra sgRNA, along with a Cas protein, binds to and cuts the newly formed strand or the ssDNA, forming a sticky end. These newly formed sticky ends facilitate ligation of the newly formed fragments (step f).

### Implementing Technologies

DNA polymerase-mediated genome editing can be carried out by transfecting target cells with polynucleotides encoding the sgRNA, ssDNA and the fusion protein or complex. Transfection is often accomplished by introducing vectors into a cell.

In some embodiments, the RNA/DNA/proteins can be introduced to a cell directly as proteins and RNA, or their complexes. Each molecule can be introduced separately, or together, without limitation.

Vectors may be introduced into the desired host cells by known methods, including, but not limited to, transfection, transduction, cell fusion, and lipofection. Vectors can include various regulatory elements including promoters. In some embodiments, the present disclosure provides an expression vector including any of the polynucleotides described herein, e.g., an expression vector including polynucleotides encoding the fusion protein and/or the sgRNA, ssDNA.

In some embodiments, the contacting occurs in the presence of a DNA repair system, which forms a double-stranded DNA sequence introduced at the target site, wherein one strand of the double-stranded DNA sequence is encoded by the first fragment, the first pairing fragment, and a reverse-complement of the second fragment collectively. Such contacting can be, for instance, in a cell, in vitro, ex vivo, or in vivo. The cell may be a prokaryotic cell, a eukaryotic cell, a plant cell, an animal cell, a mammal cell, or a human cell.

The introduced nucleic acid sequence, whether for insertion only or insertion and replacement, is at least 1 bp in length. Preferably, however, the length of the inserted or replaced sequence is at least 45 bp in length, or at least 60 bp, 80 bp, 100 bp, 150 bp, 200 bp, 250 bp, 300 bp, 350 bp, 400 bp, 450 bp, 500 bp, 600 bp, 700 bp, 800 bp, 900 bp, 1000 bp or 2000 bp in length.

Also provided are compositions, kits and packages useful for conducting DNA polymerase-mediated genome editing. In some embodiments, the composition, kit or package includes at least sgRNA and/or ssDNA useful for the editing, as described herein.

In some embodiments, the composition, kit or package includes polynucleotide (e.g., DNA) sequences that encode the sgRNA and/or ssDNA disclosed herein. The DNA sequences can be provided in a single sequence or a single vector, or in separate sequences or vectors, without limitation. The fusion protein or complex can also be provided as encoding polynucleotide sequences, in some embodiments.

### EXAMPLES

### Example 1. Development and Testing of DNA Polymerase-Mediated Editing

In this example, the instantly disclosed DNA polymerase-mediated genome editing method was used to introduce a target insertion at (a) EGFP and (b) HEK3 sites.

HEK293T cells were transfected with 3 μg Cas9-DNA polymerase plasmid, 1 μg of each sgRNA plasmid and 1 μg of each ssDNA donor using SF Cell line 4D-Nucleofector X Kit (Lonza) with 5E5 cells per well (program CM-130). Cells were harvested after 48 h after transfected, and Sanger sequencing were performed.

The insertions were confirmed by Sanger sequencing. The precise insertion region is highlighted in FIG. 7. The disturbed sequence peaks in Sanger sequencing were consistent with the accurate inserted sequence, confirming the effectiveness of the insertion.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A molecule comprising (a) a Cas protein and (b) a DNA polymerase, wherein the Cas protein is fused to the DNA polymerase or is coupled to the DNA polymerase through a covalent or ionic interaction, directly or indirectly.

2. The molecule of claim 1, wherein the Cas protein is selected from the group consisting of Cas9, Cas12, Cas13 and Cas14.

3. The molecule of claim 1, wherein the Cas protein is Cas9.

4. The molecule of claim 3, wherein the Cas9 is selected from the group consisting of SpyCas9, SaCas9, NmeCas9, FnCas9 and CjCas9.

5. The molecule of claim 3, wherein the Cas9 is a nickase, preferably Cas9 H840A.

6. The molecule of claim 1, wherein the DNA polymerase is selected from the group consisting of eukaryotic DNA polymerase family A, B, C, X and Y such as DNA polymerase α, γ, β, λ, ε, δ, κ, η, ξ, ι, θ, μ, σ, ν, Rev1, TdT, telomerase and human codon-optimized prokaryotic DNA polymerase from family Pol I, Pol II, Pol III, Pol IV, Pol V and Family D, including E. coli DNA polymerase I, DNA polymerase III, engineered DNA polymerase from virus and phage, such as codon-optimized Bacteriophage T4 DNA polymerase.

7. The molecule of claim 1, further comprising an accessory protein replication factor C (RF-C), a proliferating cell nuclear antigen (PCNA) or a DNA helicase.

8. The molecule of claim 1, further comprising a single guide RNA (sgRNA).

9. The molecule of claim 1, further comprising a single stranded DNA (ssDNA).

10. The molecule of claim 1, wherein the Cas protein is fused to the DNA polymerase.

11. The molecule of claim 10, wherein the Cas protein is located at the N-terminal side of the DNA polymerase, or at the C-terminal side of the DNA polymerase.

12. A method for introducing a foreign nucleotide sequence into a target nucleotide, comprising contacting, in a cell, the target nucleotide with a Cas protein fused or coupled to (or co-present with in the cell) a DNA polymerase, a single guide RNA (sgRNA) comprising a spacer complementary to a protospacer in the target nucleotide, a donor single stranded DNA (ssDNA) that is complementary to a portion of the target nucleotide on the opposite strand of the protospacer, and further encodes the foreign nucleotide sequence.

13. A method for introducing a foreign nucleotide sequence into a target nucleotide, comprising contacting, in a cell, the target nucleotide with a Cas protein fused or coupled to a DNA polymerase,
(a) a first single guide RNA (sgRNA) comprising a first spacer complementary to a first protospacer in the target nucleotide, a first donor single stranded DNA (ssDNA) that is complementary to a first portion of the target nucleotide on the opposite strand of the first protospacer, and further encodes a first portion of the foreign nucleotide sequence; and
(b) a second single guide RNA (sgRNA) comprising a second spacer complementary to a second protospacer in the target nucleotide, a second donor single stranded DNA (ssDNA) that is complementary to a second portion of the target nucleotide on the opposite strand of the second protospacer, and further encodes the remaining portion of the foreign nucleotide sequence.

14. The method of claim 13, wherein each of the first ssDNA and the second ssDNA further comprise a 5′ fragment complementary to each other.

15. The method of claim 14, wherein each 5′ fragment is of length of 1 to 50 bases, 2 to 40 bases 3 to 30 bases, 4 to 25 bases, 5 to 20 bases, 5 to 15 bases, or 5 to 10 bases.

16. The method of claim 13, wherein each ssDNA further comprises a spacer or a protospacer adjacent motif (PAM) 5′ to the portion that encodes the foreign nucleotide sequence.

17. The method of claim 16, wherein the cell further includes a third sgRNA that recognizes the spacer or the PAM on each of the ssDNA.

18. The method of claim 13, wherein each complementary portion of the target nucleotide and the corresponding protospacer are on opposite strands of the target nucleotide and are within 10000 base pairs from each other, or preferably within 5000, 1000, 500, 100, 90, 80, 70, 60, 50, 40, 30, 20, 15 or 10 base pairs from each other.

19. The method of claim 18, wherein the complementary portion is of length of 1 to 50 bases, 2 to 40 bases 3 to 30 bases, 4 to 25 bases, 5 to 20 bases, 5 to 15 bases, or 5 to 10 bases.

20. The method of claim 13, wherein the Cas protein is selected from the group consisting of Cas9, Cas12, Cas13 and Cas14.

21-32. (canceled)
